(19) United States
(12) Patent Application Publication
(10) Pub. No.: US 2003/0072829 A1
(43) Pub. Date: Apr. 17, 2003

(54) Bacterial isolates from organisms that respire at least partially through their skin and biologically active extracts derived therefrom

(76) Inventor: Richard M. Austin JR., Demorest, GA (US)

Correspondence Address: Kent A. Herink, Esq., The Financial Center, Suite 2500, 666 Walnut Street, Des Moines, IA, 50309, US

(21) Appl. No.: 10/037,881

(22) Filed: Jan. 4, 2002

Related U.S. Application Data
(60) Provisional application No. 60/260,022, filed on Jan. 5, 2001.

(51) Int. Cl. A61K 35/68

(52) U.S. Cl. 424/780

(57) ABSTRACT

Extracts including a biologically active compound or combination of compounds derived from microorganisms isolated from mucus-producing organisms that respire at least partially through their skin. Rod-shaped bacteria isolated from the skin of salamanders and frogs are found to produce compound(s) which have antiviral, antitumor, antibacterial and antifungal properties. These compound(s) have an inhibitory effect on opportunistic human pathogens, including Candida sp., Microsporum sp., Staphylococcus sp., Pseudomonas sp., Escherichia sp, and Enterococcus sp, as well as on HIV strains and tumor cell lines.

### PRIOR APPLICATION

This application claims priority to provisional application Serial No. 60/260,022, filed Jan. 5, 2001.

### BACKGROUND OF THE INVENTION

All amphibians respire, to varying degrees, cutaneously. As such, their integument must serve as a gas permeable barrier to their external environment (Lilleywhite, H. B., and P. F. A. Maderson. 1988. The structure and permeability of the integument. American Zoologist 28:945-962). Moisture is requisite for skin to be utilized as a respiratory organ (Fox, H. 1994. Structure of the integument. Cpt. 1 in “Amphibian Biology, Vol 1, The Integument”, ed by H. Heatwole and G. T. Barthalmus, Surrey Beatty and Sons, Chipping Norton). This necessary moisture is achieved through the production of mucus via mucus-producing glands associated with the integument (Duellman, W. E., and L. Trueb. 1986. Biology of Amphibians. McGraw Hill, New York, N.Y., U.S.A.; Fox 1994). The primary component of mucus in amphibians is a mucopolysaccharide (glycoprotein) (Duellman and Trueb 1986). Glycoproteins contain one or more carbohydrate chains covalently linked to a polypeptide backbone (Schaechter, M., and I. Brockhausen. 1989. The biosynthesis of branched O-glycans. In Mucus and related topics. E. Chantler and N. A. Ratcliffe (eds). Symposia of the Society for Experimental Biology, no. XLIII, University of Cambridge, Cambridge). The mucus is rich in carbon, a necessary element to support microbial growth and synthesis of most, if not all, cellular compounds (Guirard, B. M., and E. E. Snell. 1962. Nutritional requirements of microorganisms. Pp. 33-93. In I. C. Gunsalis and R. Y. Staneir (Eds.), The Bacteria. A Treatise on Structure and Function. Vol. IV: The Physiology of Growth. Academic Press, New York, N.Y., U.S.A.). Thus, the mucus layer necessarily produced by amphibians in order to accomplish cutaneous respiration represents a nutrient rich habitat for microorganisms (Austin, Jr., R.M. 2000. Cutaneous microbial flora and antibiosis in Plethodon ventralis: inferences for parental care in the Plethodontidae. Pp. 451-461. In R. C. Bruce, R. G. Jaeger and L. D. Houck (Eds.), The Biology of Plethodontid Salamanders, Kluwer Academic/Plenum Pub., New York, N.Y., U.S.A.).

The majority of microorganisms in most ecosystems are attached to surfaces (Wimpenny, J. W. T., S. L. Kinniment, and M. A. Scourfield. 1993. The physiology and biochemistry of biofilms. Pp. 274-318. In S. Denyer, P. Gorman, and M. Sussman, (Eds.), Microbial Biofilms: Formation and Control. Blackwell Scientific Publications, London, U.K.), and the integuments of animals often serve as suitable habitats for the development of microbial communities (Alexander, M. 1971. Microbial Ecology. John Wiley and Sons, New York, N.Y., U.S.A.). These microcommunities often exhibit the same types of community-level interactions that communities of larger organisms (macrocommunities) exhibit. Members of microcommunities compete for limited resources and form intimate, and, at times, ammensalistic relationships (Atlas, R. M., and R. Bartha. 1993. Microbial Ecology. Fundamentals and Applications, 3rd ed. Benjamin Cummings, New York, N.Y., U.S.A.; Bull, A. T., and J. H. Slater. 1982. Microbial Interactions and Communities. Vol. 1. Academic Press, New York, N.Y., U.S.A.; Frederickson, A. G., and G. Stephanopoulos. 1981. Microbial competition. Science 213:972-979). Such amensalistic strategies include, but are not limited to, the production of antibiotics.

Antibiotics are substances produced by microorganisms that are capable of killing or inhibiting growth of other microorganisms (Williams, S. T. 1982. Are antibiotics produced in soil? Pedobiology 23:85-87, Williams, S. T., and J. C. Vickers. 1986. The ecology of antibiotic production. Microbial Ecology 12:43-52; Brock, T. D. 1994. The biology of Microorganisms. 7^{th} ed., Prentice Hall, Englewood Cliffs, N.J.). The production of such substances has been viewed as an adaptation to reduce or inhibit competition in natural environments (Weiner 1996). Competition studies between antibiotic-producing and antibiotic sensitive bacteria in both solid and liquid media have provided evidence that supports this theory (e.g. Rasool, K., and j. W. T. Wimpenny. 1983. Mixed continuous culture experiments with an antibiotic-producing streptomycete and Escherichia coli. Microbial Ecology 8:267-277; Turpin, P. E., V. K. Dihr, K. A. Maycroft, C. Rowlands, and E. M. H. Wellington. 1992. The effect of Streptomyces species on the survival of Salmonella in soil. FEMS Microbiol. Ecol. 101:271-280). Laboratory experiments indicate that antibiotic production prevents invasion of competitors but does not improve ability to invade or compete with established populations of antibiotic-sensitive microbes (Wiener, P. 1996. Experimental studies on the ecological role of antibiotic production in bacteria. Evolutionary Ecology 10:405-421). Despite the evidence for antibiotic production in laboratory experiments, the natural role of antibiotics and the conditions under which they are produced remains unclear (Wiener 1996).

Salamanders of the family Plethodontidae comprise the largest family of extant salamanders. Its members are among the most numerous vertebrates in many forest and lotic ecosystems. They are characterized most notably by their lack of lungs. Life histories exhibited within the family are extremely diverse (e.g., Tilley, S. G., and J. Bernardo. 1993. Life history evolution in plethodontid salamanders. Herpetologica 49: 154-163; Wake, D. B., and S. M. Marks. 1993. Development and evolution of plethodontid salamanders: a review of prior studies and a prospectus for future research. Herpetologica 49:194-203). Despite such variations, the conservatism in their reproductive biology, particularly the near universality of parental care in the form of egg attendance (Salthe, S. N., and J. S. Mecham. 1974. Reproductive and courtship patterns. Pp. In B. Lofts (Ed.), Physiology of the Amphibia. Vol 2: Academic Press, New York, N.Y., U.S.A.), is well documented (e.g., Forester, D. C. 1978. Laboratory encounters between attending Desmognathus ochrophaeus (Amphibia, Urodela, Plethodontidae) females and potential predators. Journal of Herpetology 12:537-541, 1979; Highton, R., and T. Savage. 1961. Functions of the brooding behavior in the female red-backed salamander, Plethodon cinereus. Bulletin of the Florida State Museum 6:235-237; Piersol, W. H. 1909. The habits and larval state of Plethodon erythronotus. Transactions of the Canadian Institute 8:469-493; Ritter, W. E. 1903. Further notes on the habits of Autodax lugubris. American Naturalist 37:883-886; Tilley, S. G. 1972. Aspects of parental care and embryonic development in Desmognathus ochrophaeus (Amphibia: Plethodontidae) in western North Carolina. American Midland Naturalist 89:394-407).

Parental care can be defined as post-zygotic parental investment in progeny (Trivers, R. L. 1985. Social Evolution. Benjamin Cummings, New York, N.Y., U.S.A.). Within the Plethodontidae this investment occurs in the form of egg attendance, most often by the female parent (Crump, M. L. 1995. Parental care. Pp. 518-567. In H. Heatwole and B. K. Sullivan (Eds.), Amphibian Biology. Vol. 2: Social Behaviour. Surrey Beatty and Sons, Chipping Norton, New South Wales, Australia). In most cases the female remains with the clutch until hatching occurs. The adaptive benefits of such behavior in plethodontids have been clearly established, and include protection against predators, fungal infections from nonpathogenic common soil fungal species, desiccation, and prevention of developmental abnormalities, (see Crump, 1995, for a comprehensive review). Parental care consists of a behavioral repertoire (e.g., bodily contact with the clutch, egg manipulation, gular pulsations) which apparently confers these multiple benefits simultaneously on the developing offspring.

Although the benefits of parental care within the Plethodontidae are clearly documented, the mechanisms of how certain aspects are achieved have remained unclear until recently (e.g. Austin 2000). This situation has been particularly true with respect to the many observations over the past century regarding the reduction of fungal infections in clutches when the female is present. Such observations led to the hypotheses that either females (e.g., Noble, G. K. 1931. The Biology of the Amphibia. McGraw Hill, New York, N.Y., U.S.A.; Piersol, 1909) or their developing embryos (Highton and Savage, 1961) produce an antifungal metabolite capable of reducing or inhibiting fungal growth on attended egg clutches. Subsequent research has provided no indication for the existence of antimicrobial secretions from salamanders (Daniel, J. C., Jr., and R. W. Simpson. 1954. A negative note on antibiotics. Herpetologica 10:16; Forester, D. C. 1979. The adaptiveness of parental care in Desmognathus ochrophaeus (Urodela: Plethodontidae). Copeia 1979:332-341; Tilley, 1972; Vial, J. L., and F. B. Prieb. 1966. Antibiotic assay of dermal secretions from the salamander, Plethodon cinereus (Green). Herpetologica 22: 284-287; Vial, J. L., and F. B. Prieb. 1967. An investigation of antibiosis as a function of brooding behavior in the salamander, Plethodon cinereus. Transactions of the Missouri Academy of Science 1:37-40). No published accounts are known of researchers utilizing the cutaneous environment of amphibians as a potential source of medically-useful bacteria. Much research regarding medically useful compounds from amphibians and other organisms has focused upon the production of such compounds by amphibians and other macro-organisms in their skin secretions rather than the bacteria flora which inhabit the skin of such organisms (e.g. Glausiusz, J. 1998. The frog solution. Discover. 19: (11) 88-92; Carte, B. K. 1996. Biomedical potential of marine natural products. Bioscience. 46(4): 271-287; Port, O. 2001. A leap in drug research from frogs. Business Week. 3752:99; Tyler, M. J. 1995. Frogs and Drugs. Australian Natural History. 24(12): 46-52; Coghlan, A. 2000. Bug busters. New Scientist. 166(2233):15; Stienborner, S. T., G. J. Currie, J. H. Bowie, J. C. Wallace, and M. J. Tyler. 1998. New antibiotic caerin 1 peptides from the skin of the Australian tree frog Litoria chloris. Comparison of the activities of the caerin 1 peptides from the genus Litoria. Journal of Peptide Research, 51: (2), 121-127; Rennie, J. 1993. No snake oil here. Scientific American 266 (3) 136-137; McNamee, D. 1994. Eye of newt, toe of frog. Lancet. 344(8938):1696-1698; Erspamer 1994).

Because of the nature of the nutrient rich mucus produced to facilitate cutaneous respiration, other aspects regarding the success of plethodontid salamanders may be attributable to their resident microorganismal flora. Because plethodontid salamanders must rely solely upon their skin for the exchange of respiratory gases, excessive microbial loads of aerobic soil bacteria inhabiting the surface of the skin would provide an impenetrable barrier for respiratory gases, ultimately leading to the death of the salamander.

The nutrient rich skin environment of amphibians has been shown to support a microbial community that is distinct from those of surrounding environments (Austin, Jr., R. M. 1997. The cutaneous bacterial flora of the eastern zigzag salamander, Plethodon dorsalis Cope (Amphibia:Plethodontidae). Dissertation. University of Mississippi, University, Miss.). Given that microorganisms have been shown to utilize amensalistic strategies in nutrient rich environments, including the production of antibiotics (Frederickson and Stephanopoulos, 1981), the cutaneous environment of amphibians can be predicted to support microorganisms capable of producing compounds which reduce or inhibit microbial growth, thus imparting a competitive advantage to the ecology and life history of amphibians. Surprisingly, it has recently been determined that metabolites produced by certain of the microorganisms isolated from the cutaneous environment of organisms that respire at least partially through their skin exhibit antimicrobial activity against important human pathogenic bacteria and fungi, as well as having antiviral and antitumor activities.

### SUMMARY OF THE INVENTION

The invention consists of extracts which comprise compounds and combinations of compounds produced by microorganisms isolated from the body of mucus-producing organisms that respire at least partially through their skin, and which have antitumor, antiviral, antifungal, and/or antibacterial activity in humans. More specifically, the invention consists of antibiotics produced by bacteria growing in the mucus of animals which respire through their skin. Even more specifically, the invention consists of compounds and/or combinations of compounds produced by bacteria isolated from the skin of amphibians which have antiviral, antitumor, antibacterial and/or antifungal activity, including activity against bacteria and/or fungi which are pathogenic to humans. Specific examples include extracts which comprise compound(s) produced by bacteria isolated from the skin of salamanders and frogs which have activity against Candida sp., Microsporum sp., Streptococcus sp. (including penicillin-resistant Streptococcus), Staphylococcus sp. (including methicillin-resistant Staphylococcus), Enterococcus sp. (including vancomycin-resistant Enterococcus), Pseudomonas sp., Escherichia sp, the Human Immunodeficiency Virus (HIV) strains including the reduction of cytopathicity of HIV strains in human lymphocytes CEM and MT-4, and the tumor cells f-murine leukemia, murine mammary carcinoma, and human T-lymphocyte cells.

An object of the invention is the extraction of a compound or combination of compounds from bacterial isolates from the skin of organisms that respire at least partially through their skin that have antibacterial, antifungal, antiviral, or antitumor effects.

Another object of the invention is the identification of bacterial isolates from the skin of organisms that respire at least partially through the skin that produce compounds that are extracted from the isolates that have antibacterial, antifungal, antiviral, or antitumor effects.

### BRIEF DESCRIPTION OF THE DRAWINGS

FIG. 1 is a photograph of a petri dish used as an inhibition trial control, wherein the vertical streak (AC024) includes a bacterium isolated from the skin of Acris crepitans, the horizontal streaks are the four human bacterial pathogens Enterococcus faecalis (ATCC 29212), Escherichia coli (ATCC 25922), Pseudomonas aeruginosa (ATCC 27853), and Staphylococcus aureus (ATCC 29213), and which shows the presence or absence of an inhibitory effect based on the proximity of growth of horizontal human pathogen bacterial streaks to the vertical amphibian bacterial streaks.

FIG. 2 is a photograph of a petri dish used as an inhibition trial control as described with reference to FIG. 1, wherein the vertical streak (EC024) includes a bacterium isolated from the skin of Eurycea cirrigera.

FIG. 3 is a photograph of a petri dish used as an inhibition trial control as described with reference to FIG. 1, wherein the vertical streak (RC003) includes a bacterium isolated from the skin of Rana clamitans.

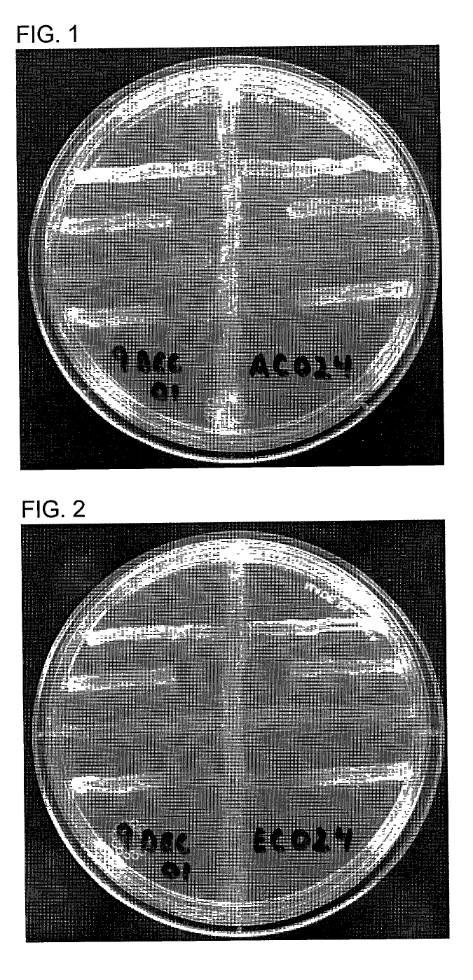

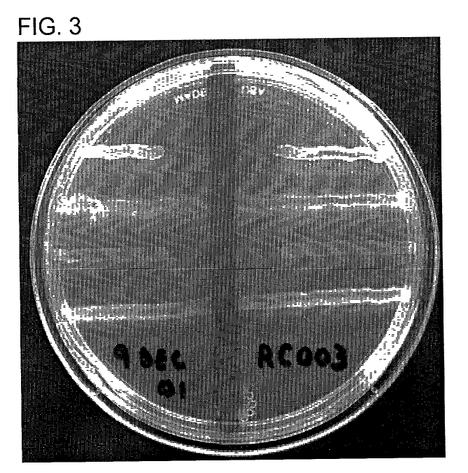

### DETAILED DESCRIPTION OF PREFERRED EMBODIMENTS

The invention comprises the identification of bacterial isolates taken from the cutaneous microbial flora of animals that respire at least partially through their skin and the extraction of a compound or combination of compounds (extract) from the isolates which has or have antibacterial, antifungal, antiviral, or antitumor effects. A methodology for identifying the isolates and a methodology for producing the extracts are described. The extract may comprise a single active compound, a combination of compounds, or one or more compounds which have a synergistic effect when found in combination with other compounds in the extract.

Source organisms are collected in their native environments and placed in isolation. After the isolation period, samples of the cutaneous microflora of the organisms are grown and isolated based on morphological characteristics. The bacterial isolates and extracts from the isolates are tested for antibacterial effects against selected bacterial strains on culture media. Liquid extraction techniques are used to prepare extracts from the bacterial isolates which are then tested for the ability to inhibit the growth of selected strains of bacteria and fungi using photometric evaluation. Components of the extracts are tested for their ability to inhibit cytopathicity in cell cultures. Components of the extracts are tested for their ability to inhibit the proliferation of carcinoma cells.

### EXAMPLE 1

### Cutaneous Microbial Flora and Antibiosis on Pathogenic Bacteria in Desmognathus quadramaculatus

A brooding female of the species Desmognathus quadramaculatus was collected at Piedmont Nature Garden and placed in a 3.79 liter plastic bag filled with leaf litter and soil taken from the locality of collection, placed in a travel cooler, and transported to the laboratory. The salamander was isolated in glass petri dishes lined with moist filter paper, and maintained at 6-7° C. for one week.

Following the isolation period, bacteria were sampled from the salamander by streaking the dorsum between the pectoral and pelvic girdles with a bacteriological loop. Bacterial samples were cultured on Tryptic Soy Yeast Extract (TSYE) agar (2 g Difco Bacto tryptic soy broth, 1 g Difco Bacto yeast extract, 12 g Difco Bacto purified agar to one liter of water) and allowed to grow for 72 h at room temperature. Morphologically distinct bacterial colonies were re-isolated on TSYE agar and allowed to grow for 72 h to obtain pure cultures. Once pure cultures were obtained, isolates were cultured on TSYE agar slants, allowed to grow for two weeks at room temperature, and stored at 5° C. until needed.

The plates were incubated at ambient temperature in the dark. The next day, not much growth was observed on the plates. Three sleeves of 20 TSYE plates per sleeve were prepared. The next day, the four plates from the brooding D. quadramaculatus female were observed to have surprisingly little growth, only seven colonies being observed on the four plates. The isolates were struck on new TSYE plates for isolation and were incubated at ambient temperature in the dark.

Upon observation the next day, six colonies were seen and identified as DQ001A, DQ001B, DQ001C, DQ001D, DQ001E, and DQ001F. DQ001D, notably, was an opaque edge, white center colony.

Six plates were prepared with parallel stripes of five known opportunistic human pathogens, Staphylococcus epidermidis, Enterobacter aerogenes, Enterococcusfaecalis, Corynebacterium xerosis, and Serratia marcescens. The plates were incubated at 37° C. for 72 hours. Each of the six plates was then struck with one of each of the six isolates DQ001A-F. The six plates were then incubated at 37° C. for 72 hours.

The six plates were then observed to determine if any of the six isolates exhibited an inhibitory effect against any of the five opportunistic human pathogens. It was observed that isolate DQ001D exhibited inhibition against S. epidermidis (inhibition zone =37 mm), E. faecalis (inhibition zone =32 mm), and C. xerosis (inhibition zone =60 mm), but no inhibition against the other two pathogens. None of the other isolates exhibited any inhibition.

### EXAMPLE 2

### Cutaneous Microbial Flora and Antibiosis on Pathogenic Bacteria and Fungi in Salamanders

### Material and Methods

Collection of Source Organisms

A series of 16 amphibian species, composed of at least five individuals per species (n=5), were collected from their natural, endemic habitats. Species and collection locations are summarized in Table 1.

TABLE 1
Collection data summary for source organisms

| Family | Species | Organism | Habitat Type |
|---|---|---|---|
| Plethodontidae | Desmognathus | Salamander | Stream Edge |
| | folkertsi | | |
| Plethodontidae | Desmognathus fuscus | Salamander | Stream Edge |
| Plethodontidae | Desmognathus | Salamander | Stream Edge |
| | quadramaculatus | | |
| Plethodontidae | Eurycea cirrigera | Salamander | Swamp |
| Plethodontidae | Eurycea guttolineata | Salamander | Stream Edge |
| Plethodontidae | Eurycea lucifuga | Salamander | Cave |
| Plethodontidae | Plethodon aureolus | Salamander | Woodland |
| | | | Forest |
| Plethodontidae | Plethodon | Salamander | Woodland |
| | chattahoochee | | Forest |
| Plethodontidae | Plethodon | Salamander | Woodland |
| | chlorobryonis | | Forest |
| Plethodontidae | Plethodon dorsalis | Salamander | Woodland |
| | | | Forest |
| Plethodontidae | Plethodon ocmulgee | Salamander | Woodland |
| | | | Forest |
| Plethodontidae | Plethodon oconaluftee | Salamander | Woodland |
| | | | Forest |
| Ambystomatidae | Ambystoma opacum | Salamander | Swamp |
| Ranidae | Rana clamitans | Frog | Swamp |
| Hylidae | Acris crepitans | Frog | Stream Edge |
| Hylidae | Pseudacris crucifer | Frog | Stream Edge |

Once collected, amphibian species were placed in 3.79 liter plastic bags and were taken from the locality of collection, placed in a travel cooler, and transported to the laboratory. Amphibian species were individually isolated in 0.0946 L plastic containers lined with moist filter paper, placed in a Coron model 6010 Environmental Chamber, and maintained at 16° C. for a period of one week.

Following the isolation period, bacteria were sampled from each amphibian species, phenotypically distinct bacteria isolated from the amphibian species were assigned a code corresponding to the individual from which the isolate was obtained. Each bacterial isolate was phenotypically characterized by standard colonial morphological techniques (Salle and Meachem, 1974). This characterization was achieved by noting whole colony forms, margin (edge) forms, elevation, color, and finish, for each distinct bacterial colony. Gram stain of the bacterial isolates was determined by the Gram stain technique as outlined in Salle, A. J. 1973. Laboratory Manual on Fundamental Principles of Bacteriology. McGraw Hill, New York, N.Y. U.S.A. to aid in identifying and classifying bacterial isolates. Cellular morphology of the bacterial isolates was also noted. Following colonial and cellular characterization, each morphologically distinct bacterium isolated from the source organisms was cultured on TSYE agar slants, allowed to grow for two weeks at room temperature, and stored at 5° C. until needed.

Agar Plate Inhibition Trials Methodology

Morphologically distinct bacterial isolates isolated from source organisms were tested for the ability to produce antimicrobial compounds effective against Gram positive and Gram negative human pathogenic bacteria. The human pathogenic bacterial species utilized in the inhibition trials were Enterococcus faecalis (ATCC #29212), Escherichia coli (ATCC #25922), Pseudomonas aeruginosa (ATCC #27853) and Staphylococcus aureus (ATCC #29213).

Agar plate inhibition trials consisted of testing each morphologically distinct bacterium isolated from source organisms for the ability to inhibit growth of the human pathogenic bacteria according to the methodology and protocol listed in Alcamo, I. E. 1994. Laboratory Fundamentals of Microbiology. Benjamin Cummings, New York, N.Y., U.S.A. Bacterial isolates from the source organisms were independently streaked on TSYE agar plates in a median streak with a bacteriological loop. The plates were then incubated at room temperatures for a minimum of 72 h to allow growth of the bacteria. The human pathogenic bacterial species were then streaked in single streaks at right angles to the initial resident bacterial culture. Care was taken to ensure that the pathogenic isolates did not touch the initial bacterial streak. The plates were then re-incubated for 48 h to allow growth of the human pathogenic bacterial species. Following incubation, the plates were examined for growth of the human pathogenic bacterial isolates adjacent to the original source orgnaisms bacteria.

Inhibition was considered as a zone of unobservable growth adjacent to the original resident bacterial streak. When observed, inhibition was quantified by measuring the distance of the zero-growth zone from the initial resident bacterium to the nearest 0.5 mm.

Antibacterial/Antifungal Metabolite Inhibition Trials Methodology

Bacteria isolated from source organisms that exhibited the ability to inhibit the growth of human pathogenic bacteria in the Agar Plate Inhibition Trials were grown in 0.5 L of liquid TSYE media at ambient temperature for a period of 7 days. Each isolate was grown in duplicate flasks for subjection to two different extraction procedures. Following the growth period, complex molecules (metabolites) produced by the bacterial isolates isolated from source organisms were extracted from the liquid media utilizing standard liquid extraction methods.

The extraction methodology consisted of two independent extraction procedures, designed to target the extraction of both water-soluble and non-water soluble compounds for further testing.

The water-soluble extraction procedure consisted of rapidly freezing the liquid media sample containing the bacterial isolate cell culture at −80° C. Following the freezing procedure, the frozen sample was lyophilized. Following lyophilization, the sample was added to 0.5 L of 100 percent methanol and mechanically stirred for one hour. The sample was filter sterilized to ensure all bacterial cell/cell fragments were removed. Methanol was then evaporated from the resulting methanol/compound mixture utilizing a rotoevaporator, leaving only the crude extract of multiple compounds containing one or more compounds with potential biological activity.

The non-water soluble extraction procedure consisted of adding 150 ml of ethyl acetate to the media sample containing the bacterial isolate cell culture. This resulting mixture was then mechanically stirred for 15 minutes. The ethyl acetate was then separated from the mixture via a separatory funnel and saved. This procedure was repeated three times, resulting in 450 ml of ethyl acetate/compound mixture. The ethyl acetate was then evaporated from the resulting mixture with the use of a rotary evaporator, leaving only the crude extract of multiple compounds containing one or more compounds with potential biological activity.

Each resulting metabolite extract obtained from both water-soluble and non-water soluble extraction procedures of each of the inhibitory bacterial isolates was then tested for the ability to inhibit one or more human pathogenic bacteria, antibiotic-resistant human pathogenic bacteria, human fungal pathogens, and a human pathogenic virus. Human pathogenic bacteria utilized were Enterococcus faecalis ATCC 29212, vancomycin-resistant Enterococcus faecium (VRE) ATCC 700221, Staphylococcus aureus ATCC 29213, Methicillin-resistant Staphylococcus aureus (MRSA) ATCC 33591, Streptococcus pyogenes ATCC 12358, penicillin resistant Streptococcus pneumonia ATCC 700674, Pseudomonas aeruginosa ATCC 27853, and Salmonella typhimurium ATCC 700408. Human pathogenic fungi utilized were Candida albicans ATCC 24433, Microsporum gypseum ATCC 14683, Trichophyton mentagrophytes ATCC 9533 and Aspergillus fumigatus IHEM 2895.

Antibacterial testing are conducted in Mueller-Hinton broth. The inoculum is prepared by making a direct saline suspension of isolated colonies selected from 18 to 24 hour agar plate. Afterwards the inoculum is standardized to 500,000 CFU/ml. The incubation temperature is 35° C. and the incubation time is 16 hours. Yeast screenings are conducted in RPMI 1640 broth supplemented with 0.3 g/l glutamine and 34.6 g/l morpholine propane sulfonic acid (MOPS-buffer). The incubation temperature is 35° C. and the incubation time is 24 hours.

The inoculum is prepared by making a direct saline suspension of isolated colonies selected from 18 to 24 hour Sabouraud agar plate. Afterwards the inoculum is standardized (1000 to 5000 CFU/ml). As measurement tool, the area under the growth curve which is automatically determined via the Biolink software. The area of the sample only containing broth and test chemical (e.g. 25 ppm of an extract) is subtracted from the area of the sample also containing inoculum. Five replicates are used for each sample, resulting in a number that can be compared with the reference antibiotic, e.g. amphotericin B, penicillin and vancomycin. Results are expressed as percentage of growth relative to the sample without test-chemical.

Antimould screenings are conducted in RPMI 1640 broth supplemented with 0.3 g/l glutamine and 34.6 g/l morpholine propane sulfonic acid (MOPS-buffer). The incubation temperature/time is 35° C./48 hours for the Aspergillus sp. The dermatophytes have an incubation temperature of 25° C. and the incubation time is 5 days.

The inoculum is prepared by covering a seven days old culture with approximately 1 ml of sterile saline. A suspension is made by gently probing the colonies with the tip of a Pasteur pipette. Afterwards the inoculum is standardized (4000 to 50000 CFU/ml). For the moulds, kinetic measurements are not used, but only beginning and end point measurements. As measurement tool we use the OD (start and end) which is automatically determined via the Biolink software. OD-start is subtracted from OD-end. Five replicates are used for each sample. These data result in a number that can be compared with the reference antibiotic, e.g. amphotericin B. Results are also expressed as percentage of growth relative to the sample without test-chemical.

The human pathogenic bacterial and fungi species were exposed to standardized concentrations of 100 and 25 micrograms/milliliter (ppm) of the metabolite extract from each of the bacteria isolated from source organisms. The metabolite inhibition trials were conducted utilizing a Bioscreen C Analyzer, an automated reader-incubator capable of monitoring turbidity development (i.e. growth) of bacterial and fungal species by vertical photometry (optical density). The results were utilized to generate growth curves in response to the metabolite extracts obtained from the bacterial isolates. For each test we also included a positive control or reference antibiotic with known MIC against the tested organism

Antiviral Inhibition Trials Methodology

Individual metabolites were extracted from the crude extracts obtained from the bacteria isolated from the skin environment of source organisms via the two extraction procedures previously described using an HPLC column, specifically, a Beckman Ultrasphere (4.6 mm×150 mm), Part No. 235330, RP-18, 5 m particle size. The eluent was a 75:25 mixture of acetonitrile and water with a flow of 1 ml/min. A UV detector was set at 210 nm. Metabolite extracts from bacteria isolated from the skin environment of source organisms, were tested for their ability to inhibit HIV-induced cytopathicity in human lymphocyte CEM and MT-4 cell cultures. CEM cells were suspended at approximately 250,000 cells per milliliter of culture medium and infected with wild-type HIV-1(III_{B}) at approximately 100 times the 50% cell culture infective dose (CCID₅₀) per milliliter. Then, 100 1 of the infected cell suspensions were added to 200-1 microtiter plate wells containing 100 1 of an appropriate dilution of the test compounds. After 4 days incubation at 37° C., the cell cultures were microscopically examined for syncytium formation. The EC₅₀ (50% effective concentration) was determined as the compound concentration required to inhibit syncytium formation by 50%.

Antitumor Trials Methodology

Metabolites extracted from bacteria isolated from the skin environment of source organisms, were tested for their ability to inhibit the proliferation of f-murine leukemia cells (L1210/0), murine mammary carcinoma cells (FM3A), and human T-lymphocyte cells (Molt4/C8 and CEM/0) cells. All assays were performed in 96-well microtiter plates. To each well were added 5−7.5×10⁴ cells and a given amount of the test compound. The cells were allowed to proliferate for 48 hr (murine leukemia L1210, murine mammary carcinoma FM3A) or 72 hr (human lymphocyte CEM and Molt) at 37° C. in a humidified CO₂-controlled atmosphere. At the end of the incubation period, the cells were counted in a Coulter counter. The IC₅₀ (50% effective inhibitory concentration) was defined as the concentration of the compound that reduced the number of viable cells by 50%.

### Results

The cutaneous microbial flora was sampled from 16 amphibian species (source organisms) comprised of 13 salamander and 3 frog species (Table 1). From these samples, a total of 417 morphologically distinct bacterial isolates were isolated. These included 356 unique isolates from salamanders comprised of 306 Gram negative rods, 20 Gram positive rods, 25 Gram negative cocci, and 5 Gram positive cocci, and 61 unique isolates from frogs comprised of 37 Gram negative rods, 10 Gram Positive rods, 7 Gram negative cocci, and 7 Gram positive cocci (Table 2).

TABLE 2
Summary of bacterial isolates isolated from the skin environment of amphibians

| | Number of | Number of | Number of Gram | Number of |
|---|---|---|---|---|
| | Gram Negative | Gram Positive | Negative Coccus | Gram Positive |
| | Rod Isolates | Rod Isolates | Isolates | Coccus Isolates |
| Salamander | 306 | 20 | 25 | 5 |
| bacterial | | | | |
| isolates | | | | |
| Frog bacterial | 37 | 10 | 7 | 7 |
| isolates | | | | |

Agar Plate Inhibition Trials

Agar plate inhibition trials of each of the 417 unique bacterial isolates against the four human bacterial pathogens Enterococcus faecalis (ATCC 29212), Escherichia coli (ATCC 25922), Pseudomonas aeruginosa (ATCC 27853), and Staphylococcus aureus (ATCC 29213), revealed the presence of 13 unique bacterial isolates capable of inhibiting the growth of one or more of the human pathogenic species. Of these 13 isolates, nine were isolated from salamanders of the family Plethodontidae and four were isolated from frogs of the families Hylidae and Ranidae. The isolates of Desmognathus quadramaculatus, in contrast to the gravid, brooding female of Experiment 1, did not exhibit substantial inhibition. It is known that the cutaneous microflora of salamanders are affected by hormones, environmental changes, and other factors, so it is not surprising that differences in the mciroflora were observed between the individual of Experiment 1 and the individuals of Experiment 2. Accordingly, the bacterial isolate DQ001D of Experiment 1 was included in the tests conducted under this Experiment 2. Colonial and cellular morphological characteristics of the 13 unique bacterial isolates capable of inhibiting pathogenic bacterial growth are summarized in Table 3.

TABLE 3
Summary of the colonial and cellular morphology characteristics of bacteria
exhibiting inhibitory capabilities isolated from amphibians

| Isolate | Colony | Margin | Elevation | | | Gram | Source |
|---|---|---|---|---|---|---|---|
| number | Form | Form | Form | Color | Finish | Reaction | Organism |
| DQ001D | Circular | Entire | Umbonate | Opaque | Glossy | Gram | Salamander |
| | | | | White | | Neg. Rod | |
| | | | | Edge, | | | |
| | | | | White | | | |
| | | | | Center | | | |
| PO014 | Circular | Entire | Convex | Peach | Glossy | Gram | Salamander |
| | | | | Center, | | Neg. Rod | |
| | | | | White | | | |
| | | | | Edge | | | |
| PO019 | Circular | Entire | Convex | Yellow | Glossy | Gram | Salamander |
| | | | | | | Neg. Rod | |
| PO026 | Circular | Entire | Convex | Opaque | Glossy | Gram | Salamander |
| | | | | White | | Neg. Rod | |
| PO027 | Circular | Undulate | Convex | White | Glossy | Gram | Salamander |
| | | | | | | Neg. Rod | |
| AC021 | Circular | Undulate | Convex | Opaque | Glossy | Gram | Frog |
| | | | | White | | Neg. Rod | |
| AC024 | Irregular | Undulate | Convex | White | Glossy | Gram | Frog |
| | | | | | | Neg. Rod | |
| PC017 | Circular | Entire | Convex | White | Glossy | Gram | Frog |
| | | | | | | Positive | |
| | | | | | | Rod | |
| PD026 | Circular | Undulate | Convex | Opaque | Glossy | Gram | Salamander |
| | | | | Peach | | Neg. Rod | |
| EG006 | Irregular | Lobate | Convex | Orange | Glossy | Gram | Salamander |
| | | | | | | Neg. Rod | |
| EC009 | Punctiform | Entire | Convex | Yellow | Glossy | Gram | Salamander |
| | | | | | | Neg. Rod | |
| EC024 | Irregular | Lobate | Umbonate | Opaque | Glossy | Gram | Salamander |
| | | | | White | | Neg. Rod | |
| | | | | Edge, | | | |
| | | | | White | | | |
| | | | | Center | | | |
| RC003 | Circular | Serrate | Convex | Purple | Glossy | Gram | Frog |
| | | | | | | Positive | |
| | | | | | | Coccus | |

Of the 13 unique bacterial isolates isolated from source organisms, eight exhibited the capability of inhibiting more than one of the human pathogenic species against which they were tested. Five of the unique bacterial isolates isolated from source organisms were effective in inhibiting only one of the human pathogenic species against which they were tested. As can be seen from the data presented in Table 4, bacterial isolates DQ001D, AC021, EG006, EC009, PO026, PO027, and RC003 exhibited the capability of producing compound(s) inhibiting the growth of Enterococcus faecalis (ATCC 29219). Bacterial isolates EG006, EC009, PO014, EC024, and AC024 exhibited the capability of producing compound(s) inhibiting the growth of Escherichia coli (ATCC 25922). Bacterial isolates AC021, PO019, PO014, PO027, RC003, PD026, and PC017 exhibited the capability of producing compound(s) inhibiting the growth of Staphylococcus aureus (ATCC 29213). Bacterial isolates EC024 and AC024 exhibited the capability of producing compound(s) inhibiting the growth of Pseudomonas aeruginosa (ATCC 27853).

TABLE 4
Inhibition zones of human pathogenic bacteria by the bacterial isolates

| | Inhibition Zone (mm) | | | |
|---|---|---|---|---|
| | | | | Pseudo- |
| | Enterococcus | Escherichia | Staphylococcus | monas |
| Isolate | faecalis | coli | aureus | aeruginosa |
| DQ001D | 37.0 | | | |
| PO014 | | 17.5 | >50 | |
| PO019 | | | 45.5 | |
| PO026 | 25.0 | | | |
| PO027 | 23.0 | | 22.0 | |
| AC021 | 27.0 | | 30.5 | |
| AC024 | | 29.5 | | 32.0 |
| PC017 | | | 22.0 | |
| PD026 | | | >50 | |
| EG006 | >50 | >50 | | |
| EC024 | | 35.5 | | 32.0 |
| EC009 | >50 | >50 | | |
| RC003 | 44.0 | | 45.5 | |

Antibacterial/Antifungal Metabolite Inhibition Trials

Metabolite extracts from the 13 bacterial isolates isolated from the skin environment of source organisms which inhibited pathogenic bacterial growth during agar plate inhibition trials were tested for their efficacy in inhibiting pathogenic bacterial or fungal growth. Of these metabolites, extracts from bacterial isolates DQ001D, PO026, PO027 and PC017 exhibited strong biological activities in inhibiting the growth of pathogenic bacteria, pathogenic fungi, or both.

The metabolite extract from isolate number DQ001D was effective in inhibiting multiple Gram positive bacterial pathogenic species at both 100 ppm (micrograms/milliliter) and 25 ppm. Specifically, metabolic extracts from DQ001D completely inhibited the growth of pathogenic species Enterococcus faecalis, vancomycin-resistant Enterococcus faecium, Staphylococcus aureus, methicillin-resistant Staphylococcus aureus, Streptococcus pyogenes, and penicillin-resistant Streptococcus pneumonia. Additionally, the metabolite extract from DQ001D was also effective in completely inhibiting the growth of the pathogenic human fungus Candida albicans at 100 ppm and showed partial growth inhibition of the human fungal pathogen Microsporum gypseum at 100 ppm.

The metabolic extract from isolate number PO026 showed complete growth inhibition of the pathogenic fungus Candida albicans at 100 ppm.

The metabolic extract from isolate number PO027 was effective in completely inhibiting the growth of the human bacterial pathogen Staphylococcus aureus at 25 ppm. Additionally, the metabolic extract from PO027 also inhibited the growth of the fungal pathogen Candida albicans at 250 ppm.

The metabolic extract from isolate number PC017 exhibited the ability to inhibit the growth of the human fungal pathogen Candida albicans at 100 ppm. Similar results of antibacterial/antifungal activities of extracts from other bacterial isolates from amphibian species have been recently obtained, further indicating the application of the invention, leading to the expectations of finding other such activities in the future.

Antiviral Inhibition Trials

Metabolites extracted from the 13 bacterial isolates isolated from the skin environment of source organisms which inhibited pathogenic bacterial growth during agar plate inhibition trials were tested for their efficacy in inhibiting the growth of multiple strains of the Human Immunodeficiency Virus (HIV). Of these extracts, the metabolite extract from DQ001D (retention time 7.27 min) exhibited marked abilities in inhibiting HIV cytopathicity in human lymphocyte CEM and MT-4 cell cultures. Specifically, the effective metabolite concentration of DQ001D required to inhibit HIV cytopathicity of HIV-1 and HIV-2 in CEM cells was 7 ppm and 17 ppm respectively. The effective metabolite concentration of DQ001D required to inhibit HIV-induced cytopathicity of HIV-1 in MT-4 cells was 9 ppm. The determined toxicity of the metabolite extract from DQ001D on MT-4 cells was 40 ppm. Based on the experience to date testing the isolates, it is expected that future antiviral activity of other isolates will be indicated.

Antitumor Trials

The metabolite extract from bacterial isolate number DQ001D was shown to inhibit the proliferation of f-murine leukemia cells (L1210/0), murine mammary carcinoma cells (FM3A) and human T-lymphocyte cells (Molt4/C8, CEM/0) at 17 ppm, 16 ppm, 17 ppm and 17 ppm respectively. Based on the experience to date testing the isolates, it is expected that future antitumor activity of other isolates will be indicated.

The resistance of bacteria and fungi to known antibiotics and antifungal drugs has led to a world-wide search for sources of new sources for drugs that are capable of combating such diseases. Because of the previous extreme low success rate of finding new bacterial sources of such medically useful compounds, researchers have focused upon macro-organisms as potential new sources of new medically useful drugs (e.g. Beattie 1992). The data presented here provide clear and substantial evidence as to amphibians being sources for medically useful bacteria capable of producing antibacterial, antifungal, antiviral, and antitumor metabolites.

Additionally, antibiotic resistance of bacteria to known drugs presents a serious world-wide health problem. This study provides clear indication that the skin environment of amphibians contains bacterial species which are capable of inhibiting the growth on multiple human bacterial and fungal pathogens, including those are highly antibiotic resistant such as vancomycin resistant Enterococcus faecium and methicillin resistant Staphylococcus aureus. Furthermore, the antiviral and antitumor activity of the metabolite extract from the bacterial isolate DQ001D provides clear indication of the application of medically useful compounds isolated from the skin environment of amphibians. Given the biological activity of this and other isolates, it is believed and expected that future isolates will exhibit similar antiviral activities.

The foregoing description comprise illustrative embodiments of the present inventions. The foregoing embodiments and the methods described herein may vary based on the ability, experience, and preference of those skilled in the art. Merely listing the steps of the method in a certain order does not necessarily constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

What is claimed is:

1. An extract from a bacterium isolated from the skin of an organism that respires at least partially through its skin that inhibits the growth of bacteria, fungi, viruses, or tumors.

2. An extract as defined in claim 1, wherein the organism is an amphibian.

3. An extract as defined in claim 1, wherein the organism is selected from the group consisting of salamanders.

4. An extract as defined in claim 1, wherein the organism is selected from the group consisting of frogs.

5. An extract as defined in claim 1, wherein the extract inhibits the growth of human pathogenic bacteria or human pathogenic fungi.

6. An extract as defined in claim 1, wherein the extract inhibits the growth of human HIV.

7. An extract as defined in claim 6, wherein the human pathogenic bacteria are selected from the group comprising Enterococcus sp., Staphylococcus sp., Escherichia sp, and Pseudomonas sp.

8. An extract as defined in claim 1, wherein the extract is effective in a range between about 5 ppm and about 250 ppm.

9. Administering to a human a therapeutic amount of an extract as defined in claim 1.

10. A bacterial isolate selected from the group consisting of DQ001D, PO014, PO019, PO026, PO027, AC021, AC024, PC017, PD026, EG006, EC009, EC024, and RC003.

11. A method of identifying biologically active bacterial metabolites, comprising culturing a bacterial isolate according to claim 10 in an assay system comprising one or more pathogenic organisms and assessing the effect of the metabolites produced by the bacterial isolate on the growth of the pathogenic organisms(s) relative to a control.

12. A method of identifying biologically active extracts, fractions, or compounds, comprising
(a) culturing a bacterial isolate according to claim 10;
(b) extracting metabolites from the culture;
(c) optionally fractionating the extracted metabolites; and
(d) assessing the ability of the extracted or fractionated metabolites to inhibit the growth of pathogenic organisms, tumor cells, or viruses in a suitable assay system.

13. A biologically active extract, fraction, or compound identified according to the method of claim 12.

14. A pharmaceutical composition comprising one or more biologically active extracts, fractions, or compounds according to claim 13 and a pharmaceutically acceptable carrier.

15. A pharmaceutical composition according to claim 14, wherein the composition is suitable for topical, enteral, parenteral, or intravenous administration to a subject.

16. A method of inhibiting the growth of pathogenic organisms in a subject comprising administering an effective amount of a pharmaceutical composition according to claim 14 to the subject.

17. A method of inhibiting the growth of tumor cells in a subject comprising administering an effective amount of a pharmaceutical composition according to claim 14 to the subject.

18. A method of inhibiting the growth of a virus in a subject comprising administering an effective amount of a pharmaceutical composition according to claim 14 to the subject.

19. A method according to claim 18, wherein the virus is a human immunodeficiency virus.
